(19) United States
(12) Patent Application Publication
(10) Pub. No.: US 2012/0077268 A1
(43) Pub. Date: Mar. 29, 2012

(54) ANIMAL-FREE CELL CULTURE METHOD

(75) Inventors: Brigitte Ghislaine Louise Aerts, Rixensart (BE); Yves Jules Maurice Ghislain, Rixensart (BE); Marie-Monique Jane Gonze, Rixensart (BE); Isabelle Solange Lucie Knott, Rixensart (BE); Carine Maggetto, Rixensart (BE)

(73) Assignee: GlaxoSmithKline Biologicals, s.a., Rixensart (BE)

(21) Appl. No.: 13/270,143

(22) Filed: Oct. 10, 2011

Related U.S. Application Data
(63) Continuation-in-part of application No. 10/547,804, filed on Sep. 2, 2005, now Pat. No. 8,034,617.

(30) Foreign Application Priority Data
Mar. 3, 2003 (GB) 0304799.0

(51) Int. Cl. C12N 5/02; C12N 5/071

(52) U.S. Cl. 435/349; 435/384; 435/358; 435/364; 435/350; 435/380

(57) ABSTRACT

The present invention relates to a process for culturing animal cells, e.g., human, diploid anchorage-dependent cells, in the absence of exogenous components of primary animal origin. In particular, the invention provides cell culture media substantially free of exogenous components of primary and secondary animal origin which comprises at least one, more preferably several, exogenous animal-free growth factors. The present invention also relates to a process for cultivating animal cells using a protease of non-animal origin for passaging cells.

### CROSS REFERENCE TO RELATED APPLICATIONS

This application is a continuation in part of U.S. patent application Ser. No. 10/547,804, filed Sep. 2, 2005, which is a national phase filing of PCT/EP04/02067, filed Mar. 1, 2004, which claims priority to GB0304799.0, filed Mar. 3, 2003, all of which are incorporated herein by reference in its entirety, and to which the application claims priority.

### FIELD OF THE INVENTION

The present invention relates to a process for culturing animal cells, such as mammalian, preferably primate, or more preferably human cells.

### BACKGROUND OF THE INVENTION

Anchorage-dependent cells, especially diploid anchorage-dependent cells, are used in a wide range of processes: for the production of health care products such as vaccines and recombinant proteins in large-scale bioprocesses, for the generation of artificial tissues used in the treatment of human injuries, for experimental investigations, for in vitro toxicology, for screening and testing of new drugs, etc.

Conventionally, anchorage-dependent cells are cultured in media containing serum or other animal-origin components as substitutes for the serum, such as bovine serum albumin (BSA) or protein hydrolysates. Serum or animal-origin components are also used during cell subcultivation and in cell cryopreservation. Serum is a major source for metabolites, hormones, vitamins, iron (transferrin), transport proteins, attachment factors (e.g. fibronectin), spreading and growth factors. It is required for the growth of many animal cells culture in vitro. In addition, serum acts as buffer against a variety of perturbation and toxic effects such as pH change, presence of heavy metal ions, proteolytic activity, or endotoxins. Albumin is the major protein component of serum and exerts several effects which contribute to the growth and maintenance of cells in culture: it acts as a carrier protein for a range of small molecules and as a transporter for fatty acids which are essential for cells but are toxic in the unbound form.

Diploid anchorage-dependent cells are routinely grown on plastic surfaces, glass surfaces or microcarriers. The cells attach and spread out by attachment factors such as fibronectin (F. Grinnel & M. K. Feld Cell, 1979, 17, 117-129). Trypsin is one of the most common animal-derived component used for cell detachment during cell passaging (M. Schroder & P. Friedl, Methods in Cell Science, 1997, 19, 137-147; O. W. Mertens, Dev Biol Stand., 1999, Vol 99, 167-180). It must be inhibited by serum or soybean trypsin inhibitor after cell detachment in order to avoid cell damage. After detachment, cells are seeded at low density on a new surface where they can multiply and form a confluent cell layer before the next subcultivation. The purpose of passaging adherent cells is to multiply and obtain a sufficient number of cells to carry out the aforementioned processes.

There are various disadvantages linked to the use of serum and of animal-derived components in these processes, including cost, batch to batch variability in their composition, their association with a higher contamination risk by adventitious agents, and the subsequent difficulties encountered in downstream processing (e.g. purification to get rid of the serum-proteins or of the introduced animal-derived proteins). Furthermore, as noted above, it is reported that serum-free media are not suitable for anchorage dependent diploid cells (O. W. Mertens, Dev Biol Stand., 1999, Vol 99, pp 167-180; O. W. Merten, Dev. Biol. 2002, 101, 233-257).

A number of low-serum or serum-free medium formulations have been developed for anchorage-dependent cell culture, in particular for diploid anchorage-dependent cell culture (M. Kan & I. Yamane, Journal of Cellular Physiology, 1982, 111, 155-162; S. P. Forestell et al. Biotechnology and Bioenineering, 1992, 40, 1039-1044). Results obtained with such media have not been satisfactory, mainly because diploid anchorage-dependent cells, which are not transformed, would need rather complex serum-free media supplemented with several growth factors and hormones, and also because production processes generally for such cells make use of serum at least during the biomass production phase (O. W. Merten, Dev. Biol. 2002, 101, 233-257). Furthermore, these media still contain components of animal origin, like BSA, protein hydrolysates, growth factors, transport proteins, amino acids, vitamins, etc. Very few attempts have been made to develop media formulations for anchorage-dependent cells which are totally free of components of animal origin. Formulations which are mostly animal-free are reported not to be able to sustain a cell growth rate equivalent to what is observed with serum and to sustain only allow a few subcultivation steps before an early senescence is observed (B. J. Walthall & R. Ham Experimental Cell Research (1981) 134 303-311). Furthermore, primary cell cultures from anchorage-dependent cells almost always involve disaggregation of cell layers or tissue using a protease, mainly a serine-protease, of animal origin, thereby involving a risk of contaminating the cell culture with adventitious virus and causing unacceptable variability in cell growth due to batch to batch variation in the enzymatic activity of the protease. For example, the use of porcine/bovine trypsin in passaging anchorage-dependent cell cultures is a well-known technique (O. W. Mertens, Cytotechnology, 2000, 34, 181-183).

There exists a need therefore, in the field of diploid anchorage-dependent cell culture, to develop a cell culture medium which is substantially free from—and preferably totally devoid of—animal-derived components, and is suitable for carrying a process for diploid anchorage-dependent cell culture with performances equivalent to that of a basal medium for the cell type supplemented with an appropriate serum, in terms of, for example, cell growth rate, senescence, cell morphology, viral or protein production.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention provides cell culture media that are substantially free from exogenous components of primary animal origin. The cell culture media of the invention comprise at least one exogeneous growth factor of non-animal secondary origin and advantageously can replace conventional culture media and serum-free media which are known to contain components from exogeneous primary and/or secondary animal origin.

Accordingly, in a first aspect, the present invention provides a cell culture medium substantially free from, and preferably devoid of, exogenous components of primary animal origin, comprising at least one, preferably more than one, exogenous growth factor of non-animal secondary origin selected from the list consisting of EGF, FGF, tri-iodo-L tyronine and hydrocortisone and at least one of IGF-1 and/or Insulin of non-animal secondary origin. Suitably said culture medium is adapted for the cultivation of animal, such as mammalian, preferably primate, or more preferably human anchorage-dependent cells, preferably diploid cells, e.g., with equivalent performance to that of a basal medium for the cell type supplemented with an appropriate serum.

Optionally the culture medium according to the invention additionally comprises a protein hydrolysate of non-animal origin. Preferably the protein hydrolysate is present. Suitably the protein hydrolysate is a wheat hydrolysate.

The invention further provides the use of the cell culture media of the invention with the use of specific proteases for passaging cells, e.g., anchorage-dependent mammalian, preferably primate, or more preferably human cells. The cells are passaged one or more times in the presence of a protease which is not from animal origin (i.e. a “non-animal” protease). In a specific aspect, the non-animal protease is a protease derived from a plant source. In yet another specific aspect, the non-animal protease is a protease derived from a bacterial source. In another specific aspect, the non-animal protease is a protease derived from a fungal source. Cell passaging with these non-animal proteases can be carried out with a level of performance equivalent to or better than that obtained with the classical process carried out using a basal medium for the cell type supplemented with an appropriate serum.

Thus, in a second aspect, the present invention relates to the use of said medium for the cultivation of animal, such as mammalian, preferably primate, or more preferably human anchorage-dependent cells, preferably anchorage-dependent diploid cells, with equivalent performance to that obtained with a basal medium for the cell type supplemented with an appropriate serum.

In a specific aspect, the invention provides a method for cultivation of animal cells, comprising 1) culturing the cells in a medium substantially free from, and preferably devoid of, exogenous components of primary animal origin, comprising at least one, preferably more than one, exogenous growth factor of non-animal secondary origin selected from the list consisting of EGF, FGF, tri-iodo-L tyronine and hydrocortisone and at least one of IGF-1 and/or Insulin of non-animal secondary origin; and 2) passaging the cells with a non-animal protease.

Surprisingly it has been determined that the media and methods according to the invention are especially adapted for culturing animal cells, such as mammalian, preferably primate, or more preferably human anchorage-dependent cells, especially anchorage-dependent diploid cells. The non-animal components used in the media and methods of the invention demonstrate equivalent or enhanced performance (e.g., cell growth rate, cell viability senescence, cell morphology, viral or protein production) to that obtained with a basal medium for the cell type, supplemented with animal-derived components such as serum. In particular, the use of the media and the non-animal protease in the cell passaging lead to enhanced cell viability, as demonstrated in decreased levels of apoptosis and necrosis.

In certain aspects, the non-animal protease is a cysteine protease. In other aspects, the non-animal protease is a serine protease. In still other aspects, the non-animal protease is part of a protease/peptidase complex, e.g., a complex containing both endoprotease and exopeptidase activities.

The invention particularly relates to a method for establishing an animal cell culture, said process comprising:

a) seeding the cells in said culture medium as herein defined,

b) allowing the cells to adhere to the substrate;

c) maintaining the cells for a desired number of cell divisions;

d) dissociating cells from the substrate with a protease of non-animal origin, thereby forming a cell suspension; and

e) placing the cell suspension and a cell culture medium of step a) in a culture device comprising an adhesion support.

The invention also provides a method of establishing an animal anchorage-dependent cell culture, comprising:

a) seeding animal cells in a culture medium which is devoid of exogenous components of primary and secondary animal origin, and which comprises exogenous components of non-animal origin comprising:
i) at least one growth factor of non-animal origin selected from EGF, FGF, tri-iodo-L-tyronine and hydrocortisone;
ii) at least one exogenous growth factor of non-animal origin selected from the group consisting of IGF-1 and/or insulin, and
iii) a non-animal protein hydrolysate; and

b) passaging said cell culture with a protease of non-animal origin.

In these methods, the protease is preferably a cysteine endopeptidase, a neutral fungal protease, a neutral bacterial protease or a trypsin-like protease. When the protease is a cysteine endopeptidase, the protease is preferably ficin, stem bromelain, or actinidin. The hydrolysate is preferably a wheat hydrolysate. Exemplary anchorage-dependant cells include AGMK, VERO, MDCK, CEF or CHO cells.

In another embodiment, the invention provides a method for maintaining an animal cell culture, said method comprising:
a) providing a culture medium as herein defined to animal cells adhered to a substrate;
b) maintaining the cells for a desired number of cell divisions;
c) dissociating cells from the substrate with a protease of non-animal origin, thereby forming a cell suspension; and
d) placing the cell suspension and a cell culture medium of step a) on a new substrate.

The invention also provides a method for establishing a culture comprising animal diploid cells, comprising:
a) seeding the cells in a culture device comprising an adhesion support and a culture medium comprising:
   i) at least one growth factor of non-animal origin selected from EGF, FGF, tri-iodo-L-tyronine and hydrocortisone;
   ii) at least one exogenous growth factor of non-animal origin selected from the group consisting of IGF-1 and/or insulin, and
   iii) a wheat protein hydrolysate;
b) allowing the cells to adhere to the substrate;
c) maintaining the cells for a desired number of cell divisions;
d) dissociating cells from the substrate with a protease of non-animal origin, thereby forming a cell suspension; and
e) placing the cell suspension and a cell culture medium of step a) in a culture device comprising an adhesion support.

The methods for establishing the cell line preferably involve repeating the steps following the seeding of the cells and the transfer to a new substrate. More preferably, these steps are repeated two or more times.

In certain aspects, the cells are harvested to produce a cell bank. In other aspects, the protease used in the methods is inactivated after treatment and prior to seeding or re-seeding.

The invention also provides a culture medium comprising
i) at least one growth factor of non-animal origin selected from EGF, FGF, tri-iodo-L-tyronine and hydrocortisone;
ii) at least one exogenous growth factor of non-animal origin selected from the group consisting of IGF-1 and/or insulin, and
iii) a wheat protein hydrolysate; and diploid anchorage-dependent animal cells. The cells are preferably mammalian cells, more preferably primate, or more preferably human anchorage-dependent diploid cells.

The invention also provides a process for maintaining an animal cell culture, said process comprising:
a) providing a culture medium as herein defined to cells adhered to a substrate;
b) maintaining the cells for a desired number of cell divisions;
c) dissociating cells from the substrate with a protease of non-animal origin, thereby forming a cell suspension; and
d) placing the cell suspension and a cell culture medium of step a) on a new substrate.

It has also been found that said process for producing cells does not require any adaptation steps before cultivating cells in the medium free from exogeneous animal-derived components and that the senescence of the cells is not affected by the absence of this adaptation step.

It is thus another aspect of the invention to provide a cell line, in particular for a animal, such as mammalian, preferably primate, or more preferably human diploid anchorage-dependent cell line, adapted for growth in a culture medium according to the invention, and in particular to provide a cell line, in particular for a animal, such as mammalian, preferably primate, or more preferably human diploid anchorage-dependent cell line, adapted for production of a biologically active product, preferably a virus, in particular a live virus for use as a vaccine.

The invention also relates to a process for the production of viruses in animal, such as mammalian, preferably primate, or more preferably human anchorage-dependent cells in a cell culture medium suitable for viral production, said medium being devoid of components of primary animal origin, and comprising at least one exogenous growth factor of non-animal secondary origin and, optionally, one protein hydrolysate of non-animal origin, said process comprising the steps of:

a) infecting the cells with the virus

b) propagating the viruses, and

c) harvesting the viruses.

The process may include submitting the harvested virus to one or more purification steps. The virus may be suitably formulated as a vaccine, with a pharmaceutically acceptable carrier, excipient and/or adjuvant.

These aspects and other features and advantages of the invention are described in more detail below.

### BRIEF DESCRIPTION OF THE FIGURES

FIG. 1. Cell density during MRC-5 cell senescence test using ficin and bromelain proteases for cell detachment and using the medium as defined in Example 1.

FIG. 2. Cell viability during MRC-5 cell senescence test using ficin and bromelain protease for cell detachment and using the medium as defined in Example 1

FIG. 3. Cell growth during MRC-5 cell senescence test using ficin and bromelain protease for cell detachment and using the medium as defined in Example 1.

FIG. 4. Comparison of cell density during MRC-5 cell senescence test obtained with the media as defined in Example 1 (individual components) and Example 2 (supplemented ultra-MEM medium).

FIG. 5. Cell viability during MRC-5 cell senescence test obtained with the media as defined in Example 1 (individual components) and Example 2 (supplemented ultra-MEM medium).

FIG. 6. Cell growth during MRC-5 cell senescence test obtained with the media as defined in Example 1 (individual components) and Example 2 (supplemented ultra-MEM medium).

FIG. 7. HAV production on MRC-5 cells multiplied by using ficin and bromelain protease for cell detachment.

FIG. 8. Cell density during cell banking of MRC-5 cells multiplied by using ficin and bromelain protease for cell detachment.

FIG. 9. Cell viability of during cell banking of MRC-5 cells multiplied by using ficin and bromelain protease for cell detachment.

FIG. 10. Cell growth during cell banking of MRC-5 cells multiplied by using ficin and bromelain protease for cell detachment.

FIG. 11. Cell density during cell banking of MRC-5 cells multiplied by using Trypzean (Prodigen, College Station, Tx) or rProtease (Invitrogen, Carlsbad, Calif.) for cell detachment.

FIG. 12. Cell viability of during cell banking of MRC-5 cells multiplied by Trypzean (Prodigen, College Station, Tx) or rProtease (Invitrogen, Carlsbad, Calif.) for cell detachment.

FIG. 13. Cell growth during cell banking of MRC-5 cells multiplied by Trypzean (Prodigen, College Station, Tx) or rProtease (Invitrogen, Carlsbad, Calif.) for cell detachment.

### DETAILED DESCRIPTION OF THE INVENTION

The practice of the techniques described herein may employ, unless otherwise indicated, conventional techniques and descriptions of organic chemistry, polymer technology, cell biology, biochemistry, which are within the skill of those who practice in the art. Specific illustrations of suitable techniques, including techniques for the preparation of pharmaceutical preparations comprising the compositions of the invention, can be had by reference to the description and examples herein. However, other equivalent conventional procedures can, of course, also be used. Such conventional techniques and descriptions can be found in standard laboratory manuals such as Butler (2004), Animal Cell Culture (BIOS Scientific); Picot (2005), Human Cell Culture Protocols (Humana Press), Davis (2002), Basic Cell Culture, Second Ed. (Oxford Press); Lanza, et al., (Eds.) (2009), Essentials of Stem Cell Biology, Second Ed. (Elsevier Academic Press); Lanza, (Ed.) (2009), Essential Stem Cell Methods (Elsevier Academic Press); Loring, et al. (Eds.) (2007), Human Stem Cell Manual (Elsevier Academic Press); Freshney (2010), Culture of Animal Cells (John Wiley & Sons); Ozturk and Hu (2006), Cell Culture Technology for Phamaceutical and Cell-Based Therapies (CRC Press); Sambrook and Russell (2006), Condensed Protocols from Molecular Cloning: A Laboratory Manual; and Sambrook and Russell (2002), Molecular Cloning: A Laboratory Manual (both from Cold Spring Harbor Laboratory Press); Stryer, L. (1995) Biochemistry, Fourth Ed. (W.H. Freeman); Nelson and Cox (2000), Lehninger, Principles of Biochemistry, Third Ed. (W.H. Freeman); and Berg et al. (2002) Biochemistry, Fifth Ed. (W.H. Freeman); all of which are herein incorporated in their entirety by reference for all purposes.

Note that as used herein and in the appended claims, the singular forms “a,” “an,” and “the” include plural referents unless the context clearly dictates otherwise. Thus, for example, reference to “an excipient” refers to one or more excipients, and reference to “the dosage regime” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

By “adapted” when used to describe a cell line is meant that the typical cell growth and cell morphology are maintained for a number of generations similar to those observed with classical media containing animal-derived components, or alternatively that the senescence is not observed significantly sooner that observed with classical media.

By “cell growth rate” is meant the average rate at which the cells grow between their thawing from a cell bank and their senescence. It is expressed in Population Doubling (PD)/day and obtained by calculating the ratio of the number of Population Doubling, observed between the cell thawing and their senescence, to the time (expressed in days) elapsed between the cell thawing and their senescence. An equivalent cell growth rate according to the invention means a cell growth rate which is at least 80%, preferably 90%, more preferably at least 95% or above, of that obtained with the cells cultivated in a basal medium for the cell type and supplemented with an appropriate serum, usually bovine serum at a 10% concentration (used as a control). Still most preferred is a cell growth rate which is higher than that obtained with cells cultivated in a serum-containing medium. By “cell morphology” is meant the morphology of the cells as assessed by optical microscopy. An equivalent performance in terms of morphology means that the cells have retained the morphology they showed when cultivated in the presence of bovine serum. As an example, MRC-5 cells will have retained their fibroblastic nature following cultivation in a medium according to the present invention.

By “senescence” is meant the loss of replicative capacity of the cells observed after a uniform, fixed number of population doubling (population doubling level, PDL), commonly termed the Hayflick limit (Harry Rubin, Nature Biotechnology, 2002, 20, 675-681). An equivalent senescence according to the invention means a senescence which is at least 70%, preferably 90%, more preferably at least 95% or above, of that obtained with cells cultivated in a basal medium for the cell type and supplemented with an appropriate serum, usually bovine serum at a 10% concentration (used as a control). Still most preferred is a senescence which occurs at a PDL higher than that observed with cells cultivated in a serum-containing medium. Typically for MRC-5 cells, which are preferred, a senescence of between about PDL60 and about PDL75 is obtained for cells cultivated in the presence of serum as described above.

By “anchorage-dependent animal cells” or “anchorage-dependent human cells” is meant cells that are either established in cell lines or cells that originate from animal or human tissues, which need a solid support for growing and multiplying normally. The solid support is basically a growth surface such as a plastic or glass surface. Example of suitable solid supports are: petri dishes, tissue culture flasks, cells factories, roller bottles or microcarriers. For the purposes of the invention the surface is not coated with any protein from animal origin nor with peptides derived from such proteins. The cells attach and spread out by attachment, i.e. by secretion of their autocrine attachment factors. Preferred anchorage-dependent cells are diploid cells. Non limiting examples of diploid anchorage-dependent cells can be found in the ATCC catalogue (WI 38: CCL-75, MRC-5: CCL-171, IMR-90: CCL-186, DBS-FRhL-2: CCL-160, MRC-9: CCL-212) or in the NIA catalog (TIG-1 and TIG-7, developed for the NIA Aging Cell Repository, TIG-1 repository number AG06173; IMR-91:191L). Preferred cells are MRC-5, WI-38, FRhL-2, MRC-9 and the most preferred cell line is MRC-5.

“Medium substantially free from” is used in reference to a medium, including a fresh and a conditioned medium, which is devoid of serum and of any exogeneous components of primary animal origin (such as BSA for example). Such a fresh medium or conditioned medium may contain traces of exogeneous components of secondary animal origin. By “medium free of components from animal origin” is meant a medium which is devoid of serum and of any exogenous components of both primary animal origin (such as BSA for example) and secondary animal origin. Exogenous components from primary animal origin comprise, for example, components from bovine (including calf), human (such as human serum albumin—HSA) or porcine origin. Components from “secondary animal origin” are defined as components which are, at one of their manufacturing steps, in contact with a product of animal origin. In particular, frequently used components from secondary animal origin are the recombinant growth factors such as insulin, EGF and FGF and IGF-1. These recombinant growth factors, which may be produced in E. coli, are in contact with bovine or porcine components used for fermentation feeding and/or for enzymating cleavages. Traces of components from secondary animal origin are in the range of less than 1%, preferably less than 0.5%, more preferably less than 0.01%, most preferably less than 0.001%, still most preferably absent (0%). Basal serum-free media and animal origin component-free media are commercially available or can be prepared by mixing each of the individual components. They are suitably supplemented with growth factors of non-animal origin. According to the present invention, preferably a medium is used which is totally free from exogenous components of animal origin. Although a medium completely free of exogenous components of animal origin is a preferred embodiment, all said components can be replaced by secondary animal origin components (such as growth factors, wheat peptone, amino acids, protease, etc as recited above) without any impact on the performance of the process.

By “animal origin” or “animal-derived” is meant mammals, e.g. humans, as well as non-mammalian animals such as insects, fish, birds, amphibians and reptiles.

The term “exogeneous” is intended to mean an externally-derived component that has been added to the medium, as opposed to a component, referred to as “endogenous”, which has been secreted by the cell. In comparison therefore, the term “endogenous” refers to a component which is synthetised and secreted (autocrine secretion) by the cell to contribute to its attachment, spreading and growth on the appropriate substrate (fibronectin, collagen, proteoglycans, growth factors, and the like) (M. R. Koller & E. T. Papoutsakis, Bioprocess Technol., 1995, 60, 61-110).

The cell culture medium of the invention is devoid of exogeneous components of primary animal origin and comprises at least one exogenous growth factor of non-animal secondary origin, preferably at least two, more preferably at least three or more growth factors. Suitably the cell culture medium comprises at least one exogeneous growth factor of non-animal secondary origin selected from the list consisting of: EGF, FGF, tri-iodo-L tyronine and hydrocortisone and at least one of IGF-1 and/or Insulin of non-animal secondary origin. Suitably the culture medium comprises a combination of EGF, FGF, tri-iodo-L tyronine and hydrocortisone of non-animal secondary origin and at least one of IGF-1 and/or Insulin of non-animal secondary origin.

The term “growth factor” refers to a protein, a peptide, or a polypeptide, or a complex of polypeptides, including cytokines, that are necessary to cell growth, that can be produced by the cell during the cultivation process, and that can affect the cell itself and/or a variety of other neighbouring or distant cells, for example, by promoting cell attachment and growth. Some, but not all, growth factors are hormones. Examplary growth factors are insulin, insulin-like growth factor (IGF), including IGF-1, epidermal growth factor (EGF), fibroblast growth factor (FGF), including basic FGF (bFGF), granulocyte-macrophage colonstimulating factor (GM-CSF), granulocyte colony-stimulating factor (G-CSF), transforming growth-factor alpha (TGF alpha), platelet-derived growth factors (PDGFs), nerve growth factor (NGF), keratinocyte growth factor (KGF), VEGF, transforming growth-factor beta (TGF beta), interleukin-8 (IL-8), interleukin 6 (IL-6), tri-iodo-L tyronine and hydrocortisone. Preferred growth factors include for example EGF, FGF (preferably bFGF), IGF-1 or Insuline, tri-iodo-L tyronine and hydrocortisone, and can be used either alone or, preferably, in combination. A preferred culture medium contains non-animal derived EGF, FGFb, IGF-1 or Insuline, tri-iodo-L tyronine and hydrocortisone. Still more preferably all components, such as those listed in Table 3, of the cell culture medium according to the invention are of non-animal primary and secondary origin.

By “protein hydrolysate” or “protein peptone” is meant, as well understood in the art, a purified preparation of a protein hydrolysate or crude fraction thereof, which is therefore protein-free. The term protein-free is intended to mean free of any functionally active protein, but may not exclude, however, non-functional peptides as may originate precisely from protein hydrolysates. A particularly suitable hydrolysate fraction contains wheat peptone protein hydrolysate, e.g., an enzymatic digest composed of peptides from a range of up to 10,000 daltons with a majority of 80% of the peptides between 300 to 1000 daltons. When present, the concentration of protein hydrolysate in the culture medium is between 0 and 10 g/L, when present preferably between 1 and 5 g/L, especially preferably 2.5 g/L. Specifically the protein hydrolysate is derived from plant (e.g. rice, corn, wheat, soya, pea, cotton, potato) or yeast. A preferred plant protein hydrolysate according to the invention is a wheat peptone protein hydrolysate.

A “fresh medium” refers to any cell culture medium, either commercially available or prepared from each of the individual components, that has not been used to cultivate any cells. According to a preferred aspect of the invention, a fresh medium is meant to refer to a commercially available medium or a medium prepared from individual components as described above. This is, according to the invention, which is devoid of primary origin animal components and has been supplemented with at least one exogenous growth factor of non-animal secondary origin as described hereinabove, and optionally, but preferably, with a protein hydrolysate of non-animal origin such as wheat protein hydrolysate.

A “conditioned medium” is intended to mean a medium that has been used by one cell culture and is reused by another. Conditioned medium includes the release of endogenous growth stimulating substances, endogenous attachment factors and specific endogenous nutrients by the first culture. It is an aspect of the invention to provide for a method for producing a conditioned culture medium comprising combining the fresh culture medium according to the invention with animal or preferably human anchorage-dependent cells to generate a conditioned culture medium.

“Culture medium”, unless otherwise specified, shall include fresh medium, conditioned medium and the mixture of both media.

### THE INVENTION IN GENERAL

The invention provides cell culture media and methods of using such media in the growth, cultivation, and establishment of animal cell cultures, e.g., mammalian cell cultures.

In a particularly preferred embodiment, the cell culture media according to the invention are substantially free from, preferably totally devoid of, exogeneous components of primary animal origin. More preferably, the cell culture media of the invention are free from exogenous animal-derived components of both primary and secondary animal origin. Suitably said medium is preferably adapted for culturing mammalian, preferably primate, or more preferably human anchorage-dependent cells, especially anchorage-dependent diploid cells. The media of the invention provides an equivalent performance in terms of, e.g., cell growth rate, cell morphology, senescence or viral production, to that obtained with a basal medium supplemented with an appropriate serum that is typically used for the cell type.

For example, a basal medium for animal cells, such as mammalian, preferably primate, or more preferably human cells, can be found in the ATCC catalog, and examples of basal media for given cell types are additionally given in Table 1. The serum used for comparative purposes is typically a bovine serum, and typically a fetal bovine serum. Thus equivalence is best assessed in comparison with a basal medium according to Table 1 containing bovine serum, typically at a concentration of 10% v/v.

TABLE 1

| Cell Type | Basal Medium* | Serum |
|---|---|---|
| MRC-5 | Minimum essential medium | Fetal bovine serum, |
| (ATCC CCL-171) | (MEM-Eagle) | 10% |
| AGMK | Minimum essential medium | Fetal bovine serum, |
| | (MEM-Eagle) or M199 | 10% |
| VERO | Minimum essential medium | Fetal bovine serum, |
| (ATCC CCL-81) | (MEM-Eagle) or M199 | 10% |
| MDCK | Minimum essential medium | Fetal bovine serum, |
| (ATCC CCL-34) | (MEM-Eagle) | 10% |
| CHO | ATCC medium Ham's F12K | Fetal bovine serum, |
| (ATCC CCL-61) | | 10% |
| WI-38 | Minimum essential medium | Fetal bovine serum, |
| (ATCC CCL-75) | (MEM-Eagle) | 10% |
| DBS-FRhL-2 | Minimum essential medium | Fetal bovine serum, |
| (ATCC CCL-160) | (MEM-Eagle) | 10% |
| MRC-9 | Minimum essential medium | Fetal bovine serum, |
| (ATCC CCL-212) | (MEM-Eagle) | 10% |
| IMR-90 | Minimum essential medium | Fetal bovine serum, |
| (ATCC CCL-186) | (MEM-Eagle) | 10% |
| IMR-91 | Minimum essential medium | Fetal bovine serum, |
| (National Institute | (MEM-Eagle) | 15% |
| of Aging—NIA) | | |

*basal medium is supplemented with amino acids and vitamins according to ATCC or NIA instructions

In a more preferred embodiment, the culture media additionally contain a non-animal derived protein hydrolysate, preferably a plant or yeast-derived protein hydrolysate.

Table 2 shows the concentration range and the preferred concentration of growth factor(s) and protein hydrolysate as added in the fresh medium. Accordingly, the exemplary concentration of growth factors in a suitable cell culture medium according to the invention is as defined in Table 2.

| | Preferred concentration | Concentration range |
|---|---|---|
| Growth factor | (mg/liter) | (mg/liter) |
| EGF | 0.005 | 0.00001-0.3 |
| FGFb | 0.003 | 0.00001-0.1 |
| T3 (triodo L-tyronine) | 0.066 | 0-1 |
| Hydrocortisone | 1 | 0-10 |
| IGF-1 | 0.1 | 0.00001-5 |
| or insulin | 5 | 0.1-1000 |
| Wheat peptone | 2500 | 0-10000 |
| Hydrolysate | | |

It will be understood that, depending on the cell-type cultured and the performance to be achieved, the fresh culture media according to the invention may be optionally further supplemented with ingredients classically found in culture media and of non-animal origin. Suitable ingredients are, for example, amino acids (including non essential), vitamins, nucleotides/nucleosides, fatty acids, antibiotics and oxidation stabilisers, which are all from non-animal origin.

Suitable fresh media are animal-free standard media such as DMEM-based (high-glucose Dulbecco's Modified Eagle's Media), MEM (Minimum Essential Medium Eagle), Medium 199, RPM-I 1640, all commercially available from, among others, Life-technologies-Gibco-BRL, BioWittaker, Sigma-Aldrich, and further adequately supplemented with growth factor(s) and optionally with a protein hydrolysate of non-animal origin as taught above. One skilled in the art will understand that the starting medium will need to be selected according to the cell-type being cultured. A preferred commercially available fresh medium is Ultra-MEM, available from BioWhittaker (cat. no 12-745F). Alternatively, depending on the cell type to be cultivated, the fresh medium is an animal-free medium prepared from each of the individual components, and comprises (list non-exhaustive) a source of carbohydrates, inorganic salts ingredients, trace of elements, amino acids (including non essential), vitamins, nucleotides/nucleosides, fatty acids, antibiotics, oxidation stabilisers and water, suitably supplemented with non-animal origin exogeneous growth factor(s) and optionally but preferably with a non-animal origin protein hydrolysate as taught above. An example of a basic composition of such a medium is given in Example I and Table 3.

The present invention also provides methods of use of the culture media as herein above described for the cultivation of cells, preferably diploid anchorage-dependent cells, more preferably eukaryotic cells, most preferably animal cells.

In particular, the invention provides methods of establishing and/or maintaining cell cultures comprising mammalian, preferably primate, or more preferably human cells. The methods of the invention provide methods for the establishment and/or maintenance of such mammalian cells in an environment devoid of exogenous animal products. The media and methods of the invention are particularly useful to provide conditions wherein mammalian cells can be maintained in culture without any animal products which may influence certain cell characteristics or otherwise cause changes to the cell biology or morphology through the introduction of animal products.

The invention also provides a cell culture composition comprising a culture medium according to the invention and diploid anchorage-dependent cells, more preferably eukaryotic cells, most preferably animal cells, such as mammalian, preferably primate, or more preferably human diploid anchorage-dependent cells.

In a preferred aspect, the invention also provides a method for producing a culture comprising animal or preferably human anchorage-dependent cells, preferably diploid cells, in a culture medium according to the invention, said method comprising:

a) seeding the cells in a culture medium of the invention, and allowing the cells to adhere to the substrate;

b) harvesting the conditioned medium resulting from step a), and detaching the cell layer from its substrate;

c) dissociating cells with a protease of non-animal origin, thereby forming a cell suspension; and

d) placing the cell suspension and the cell culture medium of step a) in a culture device comprising an adhesion support.

Steps b) to d) are optionally repeated two or more times. In certain aspects, the cells harvested from step b) are harvested to produce a cell bank, preferably after the steps b) to d) have previously been repeated at least two times. The protease used in step b) is optionally inactivated after treatment.

Depending on the cell type and on the performance of the cell culture process to be achieved, one skilled in the art will understand that the culture medium used, especially in steps a) and d), may alternatively be a fresh medium or a conditioned medium originating from a previous culture or a mixture of fresh and conditioned medium. Within the mixture, the ratio between the fresh culture medium and the conditioned culture medium is between 1:0 (100% fresh medium) and 0:1 (100% conditioned medium). The conditioned medium represents preferably from 0 to about 75% of the total volume of medium. A preferred ratio between fresh culture medium and conditioned culture medium is 1:1 (50% fresh/50% conditioned), a still more preferred ratio is between around 7:1 (87.5% fresh/12.5% conditioned) and 1:7, and a most preferred ratio is between around 3:1 (75% fresh/25% conditioned) and 1:3, and the most preferred ratio is at 3:1 (75% fresh/25% conditioned). The preferred ratios are preferably maintained throughout the culture process at every change of medium.

The protease is of non-animal origin, that is to say the protease is not purified from an animal source. The protease may be from recombinant origin, but is preferably from bacterial, yeast or plant origin, suitably from a non-animal secondary origin. A protease from recombinant origin is intended to mean any protease which is produced by recombinant DNA techniques, and involving the use of a micro-organism, e.g., bacteria, virus, yeasts, plants, etc., for its production.

Preferred proteases include: cysteine endopeptidase; neutral fungal protease (from A. oryzae); neutral bacterial protease (from Bacillus subtilis) (described in Brocklehurst, K. et al., Cysteine proteinases. In New Comprehensive Biochemistry Vol. 16, Hydrolytic Enzymes; Neuberger, A. & Brocklehurst, K., eds, pp. 39-158 (1987) Elsevier, Amsterdam); serine proteases, such as trypsin-like protease (such as rProtease, from Invitrogen, Grand Island, N.Y. catalogue number 02-106) or recombinant trypsin (such as Trypzean, from Prodigen, College Station, Tex. code: TRY). Proteases from the trypsin-like protease family are commonly found in prokaryotes, animals and viruses, surprisingly so far not found in plants. These enzymes participate in diverse physiological processes, the best known among them are digestion, fertilisation, the blood clotting cascade and developmental processes. It is thought that they diverged from a common ancestral protein. These enzymes have been extensively described in the literature (A. J. Greer, “Comparative modelling methods—application to the family of mammalian serine proteases” Proteins, Vol. 7, 317-334, 1990) and can be divided into different families bases on their structure (A. Sali & T. Blundell, “Definition of general topological equivalence in protein structures” J. Mol. Biol., 212, 403-428, 1990). A suitable protease is a serine protease such as recombinant trypsin or trypsin-like protease. A preferred protease is a neutral fungal protease or a neutral bacterial protease.

A more preferred protease for use in the invention is a cysteine endopeptidase. A particularly preferred cysteine protease is from vegetal origin. Preferred cysteine endopeptidase from vegetal origin are selected from the group consisting of: ficin (the major proteolytic component of the latex of fig, Ficus glabrata) (Liener, I. E. & Friedenson, B. Methods Enzymol, 1970, 19, 261-273), stem bromelain (extracted from the stem of the pineapple plant, Ananas comosus), actinidin (from the kiwi fruit or Chinese gooseberry Actinidia chinensis) and papain (from latex of the papaya Carica papaya fruit). Among the cysteine proteases, ficin is especially preferred.

The protease may be used in any suitable concentration so as to ensure an efficient cell dissociation (individualised cells) within a reasonable detachment time.

The process of producing diploid anchorage-dependent cells is better understood with regard to the steps as illustrated in Example 3. In brief, the cell layer originates from cells thawed and seeded for cell culture or from a previous sub-culture, in a culture medium according to the invention. Then, in a first step, for cell detachment, the medium of the anchorage-dependent cell culture is removed and kept to be used as conditioned medium for the inoculation step. The cell layer, preferably washed, is detached and dissociated in individualised cells by using a protease solution and shaking the flask. When cells are detached and individualised, the cell suspension is collected and can be used for cell inoculation or cell banking. Optionally, when the activity of the protease is toxic for the culture of the cell line, it can be inhibited with an appropriate protease inhibitor. In a second step, for cell inoculation, cells are seeded in the new flasks at the usual cell densities applied for the cell line produced. Then, culture medium, preferably a mixture of fresh culture medium and conditioned medium is added to the new flasks. In a third step, for cell growth, new cell cultures are incubated at the same temperatures and in the same atmospheres as those applied in the usual processes used for the cell line production. An optional fourth step can be applied for cell banking, after step 1 (cell detachment) and instead of steps 2 (cell inoculation) and 3 (cell growth). It is carried out by freezing cells in the medium free of animal-origin components supplemented with the usual animal origin-free cryoprotectant used for the cell line freezing (usually DMSO and methycellulose).

Usually, cells have to be adapted to the growth in a medium free of exogeneous animal-derived components, following a predetermined strategy including several cultures with decreasing concentrations of said components, before their culture in a medium totally free of components of exogenous animal origin (Chandler J P., Am Biotechnol Lab 1990, 8, 18-28). This adaptation step is required to ensure the usual cell growth and the typical cell morphology.

However, the process for producing cells according to the invention does not require any adaptation steps before cultivating cells in the medium free of components of exogenous animal origin and that the senescence of the cells is not affected by the absence of this adaptation step. This is another advantage of the invention. In fact, the usual cell growth and the typical cell morphology are maintained for a number of generations (Population Doubling) required to reach the Population Doubling Level (PDL) equal to two thirds of the PDL at which the senescence of the cells is observed. Preferably, the usual cell growth and the typical cell morphology are maintained for a number of generations (Population Doubling) required to reach the Population Doubling Level (PDL) at which the senescence of the cells is observed. The senescence of the cells is observed at a PDL equivalent to what is observed in usual processes containing animal origin components. For example, for MRC-5 cells coming from a Master Cell Bank (PDL 13) and cultivated in a medium according to the invention, the usual cell growth and the typical cell morphology are maintained during more than 50 generations (Population Doubling) after what the senescence of the cells is observed.

Accordingly the present invention also provides for a cell line, preferably an animal cell line (e.g., a mammalian, more preferably primate), and preferably a diploid anchorage-dependent cell line adapted for growth in a culture medium according to the invention. Further, the present invention also provides for a cell line, preferably an animal such as mammalian, preferably primate, or more preferably human diploid anchorage-dependent cell line adapted for production of a biologically active product, preferably a virus, in a culture medium according to the invention.

Accordingly, in another embodiment, the present invention provides a method of producing an animal cell culture, such as a mammalian, preferably primate, or more preferably human diploid anchorage-dependent cell culture for recombinant protein or virus production in a culture medium according to the invention, said method comprising passaging said cell culture with a protease as defined above. In particular, anchorage-dependent cells, typically diploid cells are seeded at low density in a nutrient medium substantially free from exogenous components of animal origin, and after they have multiplied to form a confluent layer or multilayer, they are detached to form a suspension and reseeded at low density. The protease used to detach and passage the cells is from a non-animal origin or from a recombinant origin. Exemplary proteases for such use include, but are not limited to, cysteine endopeptidases, such as ficin, stem bromelain and actinidin; a neutral fungal protease; a neutral bacterial protease; and a trypsin-like protease, e.g., Trypzean or recombinant trypsin such as rProtease. Among the cysteine proteases, ficin is especially preferred.

In a specific embodiment, the invention relates to a process for the production of viruses in an animal cell culture, such as a mammalian, preferably primate, or more preferably human diploid anchorage-dependent cell culture, comprising: a) infecting the cells with virus; b) propagating the viruses; and c) harvesting the viruses.

Optionally the harvested virus is submitted to one or more purification steps. It is a further aspect of the present invention to provide for a virus produced as herein described and formulated, as an immunogenic composition such as a vaccine, in admixture with a pharmaceutically acceptable carrier, excipient and/or adjuvant.

Depending on the cell type and on the performance of the viral production process to be achieved, one skilled in the art will understand that the culture medium used to seed the cells in step a) may alternatively be a fresh medium or a conditioned medium originating from a previous culture or a mixture of fresh and conditioned medium. Preferably, for optimal viral production, the ratio between fresh culture medium and conditioned culture medium is between 1:0 (100% fresh medium) and 0:1 (100% conditioned medium). The conditioned medium represents preferably from 0 to about 75% of the total volume of medium. Preferred ratio between fresh culture medium and conditioned culture medium is 1:1 (50% fresh/50% conditioned), still more preferably around 7:1 (87.5% fresh/12.5% conditioned) and most preferably around 3:1 (75% fresh/25% conditioned). A ratio between fresh culture medium and conditioned culture of 1:0 (100% fresh medium) is particularly preferred. The medium used to infect cells and propagate virus may be identical to the growth culture medium, more preferably it comprises 25% w/v EGF, 25% w/v bFGF and 25% w/v T3, and is optionally further supplemented with 20% w/v protein hydrolysate, preferably wheat peptone E1 (Organotechnie SA, France). Still most preferably the medium does not contain any protein hydrolysate.

The process of viral production is better understood with regard to the steps as illustrated in Example 4. Briefly, in a first step, the anchorage-dependent cells are cultured according to the processes using a medium of the invention. The cells are infected with the appropriate virus either simultaneously with the addition of the medium of the invention, or following exposure of the cells to the medium of the invention. The infection can be carried out using a number of conventional techniques, as will be apparent to one skilled in the art upon reading the present disclosure.

Following infection, infected cells are incubated at the appropriate temperature and atmospheric pressure to promote viral propagation. Following propagation, the virus is harvested after the levels of virus production have reached the appropriate levels. The method of virus harvest is according to the method routinely applied in the processes for the virus harvest. For general culture conditions applied to viral production, see Hepatitis A virus culture process (WO 95/24468), Hepatitits A virus vaccines (WO 94/06446; A. Hagen J., 2000, Bioprocess Engineering 23, 439-449).

Examples of viruses and human viral vaccines that can be produced using the medium and the process according to the present invention include live, attenuated, inactivated, recombinant modified viruses. In particular, attenuated viruses for vaccine use that can be propagated on anchorage-dependent cells include, but are not limited to: adenoviridae (i.e. adenovirus 149), herpesviridae (i.e. herpesvirus HSV, cytomegalovirus CMV, Varicella Zoster virus VZV, Epstein-Barr virus EBV), flaviviridae (i.e. dengue virus, Hepatitis C virus HEV, Japanese encephalitis virus, Yellow fever virus), Poxyiridae (i.e. Cowpox virus, Monkeypox virus, vaccinia virus, Variola virus), Picornaviridae (i.e. echovirus, coysackieviruses, Hepatitis A virus, Polioviruses, Rhinoviruses), reoviridae (i.e. rotavirus, Colorado tick fever virus), togaviridae (i.e. Eastern equine encephalytis virus, Rubella virus), hepadnaviridae (i.e. Hepatitis B virus), Retroviridae (i.e. Immuno deficiency viruses HIV/SIV, paramyxoviridae (i.e. Measles virus, Mumps virus, Parainfluenza viruses, Respiratory Syncytial virus RSV), rhabdoviridae (i.e. Rabies virus, Vesicular Stomatitis virus), Orthomyxoviridae (i.e. influenza viruses), unclassified viruses (i.e. Hepatitis E viruse, Hepatitis delta virus), astroviridae (i.e. astrovirus), coronaviridae (i.e. coronavirus), arenaviridae (i.e. Junin virus), Bunyaviridae (i.e. rift valley fever virus). In another embodiment, the production of viral vaccines using the process according to the invention include the production of recombinant proteins expressed in adherent cells.

Preferred anchorage-dependent cells include for example AGMK, VERO, MDCK (canine epithelial kidney cell), CEF (Chicken, Embryo Fibroblast) and CHO (chinese ovary) cells, and more particularly preferred cells are anchorage-dependent diploid cells such as for example MRC-5, WI-38, TIG-1, TIG-7, FRhL-2, MRC-9, IMR-90 and IMR 91. MRC-5 is a particularly preferred cell line. The process according to the invention has proven successful for the production of hepatitis A virus, Mumps virus and VZV.

According to a preferred aspect of the invention, cells infected with any of the following viruses are preferred: hepatitis, especially HAV, polio virus, HSV, especially HSV-1 and HSV-2, CMV, EBV, rubella virus, paramyxoviridae (i.e. Measles virus, Mumps virus, Parainfluenza viruses, Respiratory Syncytial virus RSV), VZV.

On average, 15 generations are required to start a master cell bank and 10 generations are required to produce a working cell bank. At least approximately 15 generations are required in order to carry out an average batch culture on the 400 L scale. Starting with an anchorage-dependent cell line and using the medium according to the present invention, it is possible to follow the same plan to prepare a master cell bank (MCB) with approximately 15 generations and a working cell bank (WCB) with approximately 10 generations, and hence a culture with approximately 15 generations under conditions developed with the medium free from exogeneous components of animal-origin.

The present invention further relates to a virus population obtainable by the method as herein defined. It further relates to a method to produce a viral vaccine, comprising admixing said virus population with a pharmaceutically acceptable carrier, excipient or adjuvant.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

### Preparation of a Fresh Medium from Individual Components

An exemplary fresh culture medium comprises all or most of the common ingredients as listed in Table 3. According to the invention it may be suitably supplemented with the growth factors and protein hydrolysate as listed in table 2.

TABLE 3
Medium free from components of animal origin

| | | Preferred |
|---|---|---|
| | Concentration | Concentration |
| | Ranges | Ranges |
| Component | mg/L | mg/L |
| NaH₂PO₄•H₂O | 60-280 | 80-150 |
| NaH₂PO₄ | 20-400 | 25-50 |
| NaCl | 5000-8000 | 6000-7000 |
| KCl | 180-600 | 250-400 |
| AgNO₃ | 0.000005-0.00004 | 0.000010-0.000060 |
| AlCl₃•6H₂O | 0.000001-0.001 | 0.00008-0.00080 |
| Ba(C₂H₃O₂)₂ | 0.000001-0.002 | 0.00002-0.00003 |
| CaCl₂ | 100-760 | 150-250 |
| CdCl₂•2½H₂O | 0.000001-0.03 | 0.000009-0.00003 |
| CoCl₂•6H₂O | 0.000001-0.0003 | 0.000001-0.00003 |
| Cr₂(SO₄)₃•XH₂O | 0.0000003-0.0004 | 0.0000005-000008 |
| (±15 H₂O) | | |
| CuSO₄•5H₂O | 0.000001-0.006 | 0.000009-0.0008 |
| Fe(NO₃)₃•9H₂O | 0.005-1 | 0.1-0.5 |
| FeSO₄•7H₂O | 0.02-2 | 0.1-0.4 |
| GeO₂ | 0.000001-0.0008 | 0.00001-0.0001 |
| H₂SeO₃ | 0.0001-0.02 | 0.0009-0.004 |
| Na₂SeO₃ | 0.001-0.02 | 0.009-0.015 |
| KBr | 0.0000001-0.0003 | 0.0000009-0.000003 |
| KI | 0.0000001-0.00009 | 0.000001-0.000004 |
| MgCl₂ | 5-150 | 10-50 |
| MgSO₄ | 20-150 | 50-100 |
| MnSO₄•H₂O | 0.000001-0.005 | 0.00001-0.00009 |
| NaF | 0.000001-0.05 | 0.000009-0.00009 |
| Na₂SiO₃•9H₂O | 0.001-0.20 | 0.01-0.1 |
| NaVO₃ | 0.00001-0.2 | 0.0001-0.0009 |
| (NH₄)₆Mo₇O₂₄•4H₂O | 0.00001-0.002 | 0.00009-0.0009 |
| NiSO₄•6H₂O | 0.000001-0.0002 | 0.000009-0.00009 |
| RbCl | 0.000001-0.0008 | 0.000009-0.00009 |
| SnCl₂•2H₂O | 0.000001-0.0009 | 0.000009-0.00009 |
| ZnSO₄•7H₂O | 0.01-0.6 | 0.09-0.4 |
| ZrOCl₂•8H₂O | 0.000001-0.005 | 0.000009-0.00005 |
| L-Alanine | 5-50 | 10-25 |
| L-Arginine•HCl | 60-500 | 100-150 |
| L-Asparagine•H₂O | 2-80 | 2-50 |
| L-Aspartic Acid | 5-90 | 10-50 |
| L-Cysteine HCL•H₂O | 0.1-30 | 1-20 |
| L-Cystine•2HCl | 25-130 | 25-50 |
| L-Glutamic Acid | 6-50 | 20-35 |
| Glycine | 7-60 | 15-50 |
| L-Histidine•HCl•H₂O | 15-70 | 20-50 |
| L-Isoleucine | 10-200 | 20-100 |
| L-Leucine | 30-200 | 50-100 |
| L-Lysine•HCl | 30-240 | 50-100 |
| L-Methionine | 2-60 | 10-25 |
| L-Phenylalanine | 2-45 | 10-45 |
| L-Proline | 2-45 | 10-45 |
| L-Serine | 2-50 | 10-40 |
| L-Threonine | 20-150 | 20-100 |
| L-Tryptophan | 3-25 | 5-15 |
| L-Tyrosine•2Na•2H₂O | 5-150 | 10-100 |
| L-Valine | 5-150 | 20-100 |
| D-Calcium Pantothenate | 0.01-3 | 0.9-2 |
| Folic Acid | 0.01-20 | 0.9-5 |
| Pyridoxal•HCl | 0.001-4 | 0.001-0.02 |
| Vitamin A (Retinol) | 0.01-0.1 | 0.01-0.09 |
| Acetate | | |
| Vitamin B (Nicotinic | 0.001-0.1 | 0.009-0.09 |
| Acid) | | |
| Vitamin B1 | 0.001-20 | 0.8-5 |
| (Thiamin)•HCl | | |
| Vitamin B2 (Riboflavin) | 0.001-5 | 0.01-0.5 |
| Vitamin B6 | 0.001-5 | 0.8-3 |
| (Pyridoxine)•HCl | | |
| Vitamin B12 | 0.001-5 | 0.7-1 |
| (Cyanocobalamin) | | |
| Vitamin C | 0.001-30 | 0.01-0.09 |
| (Ascorbic Acid) | | |
| Vitamin D2 (Calciferol) | 0.001-0.1 | 0.01-0.07 |
| Vitamin E | 0.0001-0.1 | 0.001-0.009 |
| (alpha-tocopherol) | | |
| Vitamin H (D-Biotin) | 0.0001-0.5 | 0.001-0.009 |
| Vitamin K3 (Menadione) | 0.0001-0.5 | 0.001-0.009 |
| Thymidine | 0.01-5 | 0.09-2 |
| Adenosine 5′ Triphosphate | 0.01-10 | 0.1-5 |
| disodium | | |
| Adenosine-5-phosphate | 0.001-0.2 | 0.01-0.1 |
| 2-Deoxyribose | 0.01-10 | 0.1-5 |
| D-glucose | 1000-4000 | 1500-3000 |
| Ribose | 0.01-0.9 | 0.09-0.5 |
| Lipoic acid (Thioctic acid) | 0.001-0.7 | 0.01-1 |
| Lineolic acid | 0.001-0.3 | 0.01-0.1 |
| Adenine•H₂SO₄•H₂O | 1-10 | 2-6 |
| Choline Chloride | 0.1-10 | 2-6 |
| Ethanolamine HCl | 0.1-6 | 1-4 |
| Ethanolamine | 0.0001-0.001 | 0.0001-0.0009 |
| | μl/L | μl/L |
| Glutathione | 0.001-0.1 | 0.009-0.08 |
| Guanine•HCl | 0.01-0.6 | 0.09-0.3 |
| Hypoxanthine | 0.01-15 | 0.09-5 |
| Hypoxanthine Na | 0.01-6 | 0.09-5 |
| I-Inositol | 0.6-20 | 2-10 |
| Na Pyruvate | 10-150 | 60-120 |
| Nicotinamide/Niacinamide | 0.1-15 | 0.9-4 |
| Para-aminobenzoic acid | 0.001-0.3 | 0.01-0.1 |
| Phospho-Ethanolamine | 0.1-3 | 0.9-2 |
| Putrescine•2HCl | 0.001-0.09 | 0.01-0.06 |
| Sodium acetate | 10-50 | 15-35 |
| Thymine | 0.01-0.4 | 0.05-0.3 |
| Uracil | 0.01-0.4 | 0.05-0.3 |
| Xanthine Na | 0.01-0.5 | 0.08-0.3 |
| Glutamine | 50-300 | 100-300 |
| NaHCO₃ | 1000-2500 | 1000-1500 |
| HEPES | 1700-7000 | 3000-6800 |
| Ferric fructose stock | 50-1000 | 80-200 |
| solution | μl/L | μl/L |
| Plant or yeast derived | 0-10000 | 1000-4000 |
| hydrolysate, preferably | | |
| wheat peptone | | |

Ferric fructose stock solution

| Component | Concentration* mg/L |
|---|---|
| FeCl₃•6H₂O | 2420 |
| D-Fructose | 160000 |

*In Table 3 above and iron complex (ferric fructose) is also used as an iron source in addition to an inorganic iron.

### Example 2

### Preparation of a Fresh Medium from a Commercially Available Medium Suitably Supplemented

Commercially available medium: Ultra-MEM cat. No 12-745F (Reduced Serum Medium, Protein-free Basal Medium, without L-Glutamine) available from BioWhittaker. The basal medium formulation was free from components of animal-origin but was classically designed, according to the manufacturer's instruction, to be supplemented with a small quantity of serum (such as less than 10%) and other additives (ITES=Insulin (animal origin)+Transferrin (animal origin)+Ethanolamine+Selenium). The medium has been used in the absence of the recommended supplements from animal origin (serum and ITES).

This medium has been supplemented with the following ingredients, all free from components of primary and secondary animal origin: IGF-1:0.1 mg/L; EGF: 0.005 mg/L; bFGF: 0.003 mg/L; Triiodo-L-tyronine (T3): 0.066 mg/L; Wheat Peptone E1:2.5 g/L; and further with Ferric Fructose: 0.1667 ml/L and Sodium Pyruvate: 0.055 g/L.

The following ingredients have also been added in order to optimise the culture process carried out in the absence of components of animal-origin: Glutamine: 0.2922 g/L; Glucose: 0.33 g/L Selenium (Na₂SeO₃): and 0.01 mg/L Ethanolamine: 0.0006 μl/L.

MRC-5 cells from an animal-free cell bank (PDL 21) were thawed and cultivated according to the process disclosed in Example 3 and 5, using the medium described above and the following sub-culture scheme: D7: cell inoculation by ratio 1/8 in 100 ml of growth medium composed of 12.5% of conditioned medium; D12: cell inoculation by ratio 1/4 in 100 ml of growth medium composed of 25% of conditioned medium; D16: cell inoculation by ratio 1/8 in 100 ml of growth medium composed of 12.5% of conditioned medium; D21: repeat the scheme starting at D7.

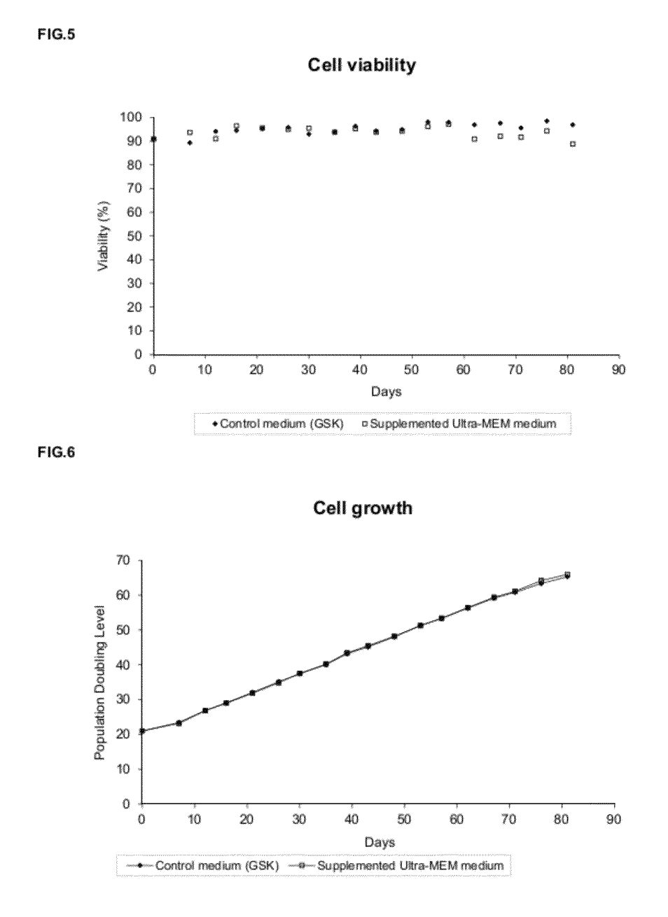

Cells were cultivated in 175 cm² T-flasks until senescence (±PDL 65) during ±3 months (e.g. 80 days). In this procedure, the cell inoculum was not fixed to a targeted cell density. Cell countings, carried out for control, show that the cell inoculum densities were between 9000 cells/cm² and 40000 cells/cm² before senescence is observed. The MRC-5 cells reached the PDL66 after 81 days of culture with a cell growth rate of 0.57 PDL/day after what senescence was observed. These results, illustrated in FIGS. 4, 5 and 6, are equivalent to what is observed with a medium prepared from individual components which leads to senescence at around PDL 65 after 81 days and cell growth rate of around 0.56 PDL.

In parallel, cells derived from this culture were used to produce HAV according to the process described in Example 4, using the same medium as described here above except that the EGF, bFGF and T3 concentrations were reduced to 25% of the concentration present in the cell growth culture medium and the wheat peptone concentration was reduced to 0.5 g/L. Harvest of virus was carried out 2 months after the start of the culture.

### Example 3

### Process for Producing Animal or Human Anchorage-Dependent Cells in a Culture Medium Substantially Free of any Components from Animal Origin

Step 1: Cell Detachment

The culture medium of an anchorage-dependent cell culture, grown in cell culture flask, was removed and kept in a sterile container. This recovered medium was considered a conditioned medium and was used for the inoculation of the cells. The cell layer was washed twice with a Phosphate Buffer Saline (PBS) supplemented with EDTA. A target of about 0.04 grams to about 1 grams of EDTA per liter of PBS and preferably about 0.2 grams/L is desirable.

Once the cell layer was washed, a sufficient volume of the protease solution was added so that the whole cell layer is covered. A targeted volume of about 0.01 ml/cm² to 2 ml/cm² and preferably 0.0333 ml/cm² is desirable. This protease solution was prepared by dissolution of the enzyme in a PBS supplemented with EDTA. A target of about 0.02 grams to about 0.5 grams of EDTA per liter of PBS and preferably about 0.1 grams/L is desirable. The quantity of protease added to the PBS/EDTA was the quantity required to generate a solution with a sufficient proteolytic activity to achieve an efficient cell detachment. The cell detachment was considered as efficient when a majority of the cells were detached from the flask and when cell aggregates were dissociated into individualized cells after a desirable targeted time of about 5 minutes to about 30 minutes and preferably about 12 minutes. The enzymatic activity of some proteases that can be used on anchorage-dependent cells is given in the following list.

A targeted enzymatic activity of about 5.5 pUPABA/ml to about 550 μUPABA/ml and preferably about 55 pUPABA/ml is desirable for Ficin (one unit of PABA is the activity of the enzyme which hydrolyzes 1 mmole of Na-benzoyl-DL-arginine-p-nitroaniline/minute at 37° C. (Methods in Enzymology Vol XIX Proteolytic enzymes p261-284).

A targeted enzymatic activity of about 0.001 Gelatin Digested Units (GDU)/ml to about 0.1 GDU/ml and preferably about 0.01 GDU/ml is desirable for Bromelain (one unit of GDU activity is the activity of the enzyme which liberates 1 mg of amino acids from a determined substrate of gelatine in the condition for the assay—(same reference as above).

A targeted enzymatic activity corresponding to a protein quantity of about 1.25 μg/ml to about 125 μg/ml and preferably about 12.5 μg/ml is desirable for neutral fungal protease from A. oryzae (according to the manufacturer, Lyven Zac Normandial, Colombelles, France).

A targeted enzymatic activity corresponding to a protein quantity of about 15 μg/ml to about 1.5 mg/ml and preferably about 150 μg/ml is desirable for neutral bacterial protease from B. subtilis (according to the manufacturer, Lyven Zac Normandial, Colombelles, France).

A targeted enzymatic activity of about 100 USP/ml to 0.1 USP/ml and preferably 1 USP/ml is desirable for Trypzean (according to the manufacturer Prodigen, College Station, Tex.).

A targeted dilution of the stock solution of about 3 times to 300 times and preferably 30 times is desirable for the rProtease (according to the supplier Invitrogen, 3175 Staley Road, Grand Island, N.Y. 14072. Supplier catalogue number 02-106).

When cell detachment was observed, the flask was gently shaked and the cell suspension is collected in a sterile container. In order to recover a maximum of cells, the flask was rinsed with fresh culture medium which was collected in the same sterile container. The cell suspension was then ready for the cell inoculation step or the cell banking step.

Step 2: Cell Inoculation

Anchorage-dependent cells obtained after cell detachment described in the step 1 were inoculated in new flasks following these instructions:

Cells were inoculated at the same cell densities as those applied in the usual processes for anchorage-dependent cell cultures with animal-origin components. For example, MRC-5 cells were inoculated at a targeted cell density of about 5000 cell/cm² to about 40000 cell/cm² and preferably between 7500 cell/cm² and 25000 cell/cm².

The volume of the growth medium added into the flask, after cell inoculation, was the same as the volume added in the usual processes for anchorage-dependent cell culture with animal-origin components. The growth medium was composed of a mixture of fresh culture medium and conditioned medium. The conditioned medium was the cell culture medium recovered at the beginning of the cell detachment step (see step 1). The quantity of conditioned medium added to the fresh medium is dependent on the cell line inoculated. A general target of 0% to about 75% of conditioned medium is desirable. To give an example, for MRC-5 cell culture, a target of about 10% to about 35% of conditioned medium is desirable and a target of about 0.025 ml/cm² to about 3 ml/cm² of culture medium added into the flasks is desirable.

Step 3: Cell Growth

Anchorage-dependent cells inoculated in a cell culture flask were incubated at the same temperatures as those applied in the usual processes for anchorage-dependent cell cultures with components of animal origin. For example, a target temperature of about 30° C. to about 40° C. and preferably at 37° C. is desirable for MRC-5 cells incubation. Anchorage-dependent cells inoculated in cell culture flasks were incubated in the same atmospheres as those applied in the usual processes for anchorage-dependent cell cultures with animal-origin components. For example MRC-5 cells can be incubated with or without CO₂ control and with or without relative humidity control.

Step 4: Cell Banking

Anchorage-dependent cells obtained after cell detachment described in the step 1 can be frozen for cell banking, following the same procedures as those applied in the usual processes for anchorage-dependent cell cultures with animal-origin components, with the following exceptions: cells must be frozen in the medium free of animal-origin components, supplemented with the same animal origin-free cryoprotectant additives as those used in the usual processes for anchorage-dependent cell freezing with animal-origin components. For example, MRC-5 cells are frozen in the medium free of animal-origin components supplemented with a desirable target of about 2.5% to about 12.5% of DMSO and a desirable target of about 0.01% to about 1% of methylcellulose.

### Example 4

### Process for the Production of Viruses in Animal or Human Anchorage-Dependent Cells in a Culture Medium

Step 5: Viral Infection

Anchorage-dependent cells are infected with the same Multipicity Of Infection (MOI) as those applied in the usual processes for anchorage-dependent cell cultures with animal-origin components. For example, a MOI target of about 0.005 to about 1 is desirable for MRC-5 cells infection by Hepatitis A Virus (HAV). Cells are infected in a medium free of animal-origin components as herein described and supplemented with ingredients according to Table 2. For the viral production, the protein hydrolysate is optional.

Step 6: Viral Propagation

Virally-infected anchorage-dependent cells (animal component free) are incubated at the same temperatures as in the usual processes used for viral propagation on anchorage-dependent cell cultures with animal-origin components. For example, a target temperature of about 31° C. to about 33° C. and preferably at 32° C. is desirable for HAV propagation on MRC-5 cells. Anchorage-dependent cells infected are incubated in the same atmospheres as those applied in the usual processes for viral propagation on anchorage-dependent cell cultures with animal-origin components. For example MRC-5 cells infected by HAV can be incubated with or without CO₂ control and with or without relative humidity control.

Step 7: Virus Harvest

The time for viral propagation between viral infection of anchorage-dependent cells and virus harvest is the same with viral propagation on anchorage-dependent cell cultures with animal-origin components. For example HAV propagation on MRC-5 cells is achieved by about 21-29 days after viral infection.

The method of virus harvest is the same as the method applied in the usual processes for virus harvest on anchorage-dependent cell cultures with animal-origin components. For example, the harvest of HAV produced on MRC-5 cells starts with two washings of the cell layer with PBS after which the virus is recovered by cell detachment using a PBS supplemented with 0.1 to 1 g/L of EDTA and then cell lysis by freezing.

### Example 5

### MRC-5 Cell Culture Until Senescence Using Ficin Protease for Cell Detachment

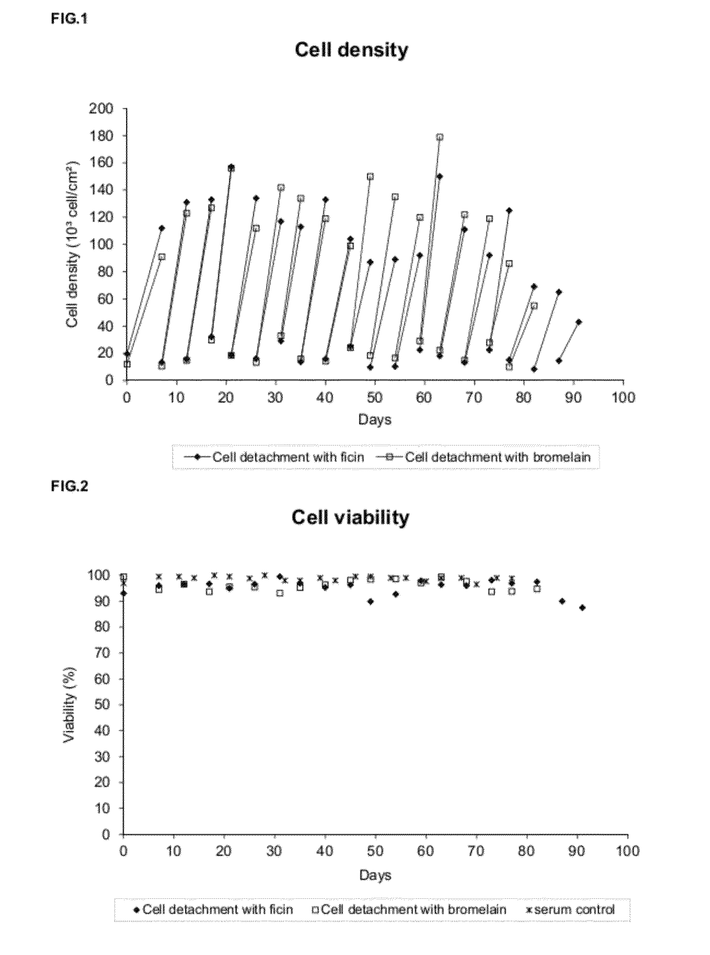

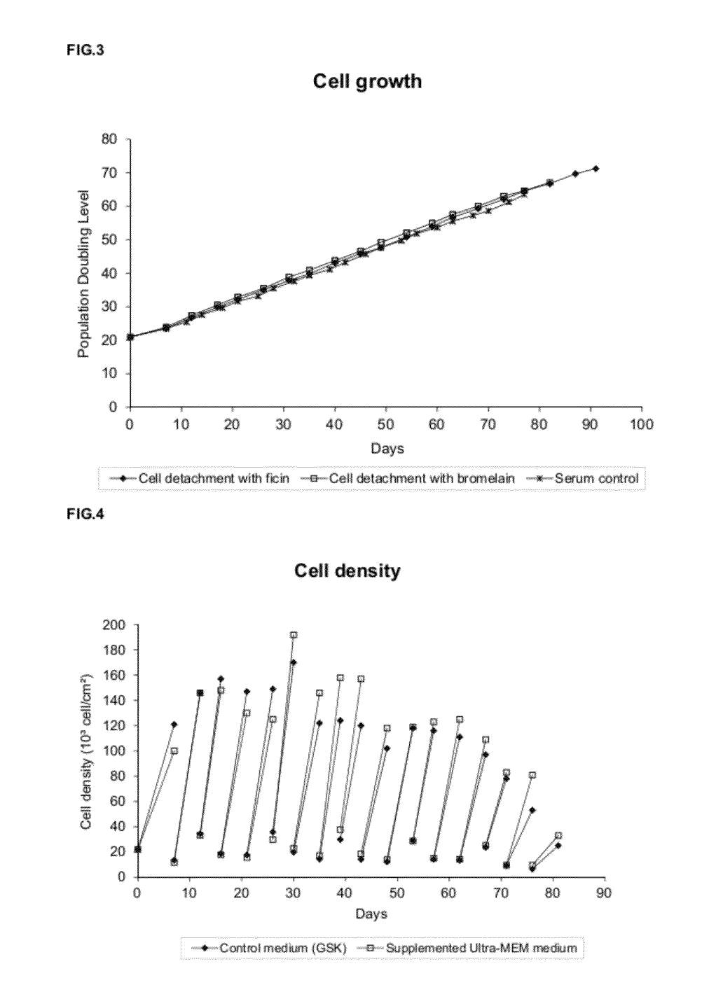

A small scale procedure for MRC-5 cells senescence testing repeated the cell production method with the process free of animal-origin components described in the steps 1 to 3 until senescence was observed. (See FIGS. 1, 2 and 3). MRC-5 cells coming from a cell bank PDL 21: free of components from animal origin were thawed, inoculated in a Nunc T175 cm² flask with 100 ml of a fresh medium suitably supplemented as described in Table 2 and incubated at 37° C. After seven days, sub-cultures (see steps 1 to 3) were carried out in Nunc T-175 cm² flask at 37° C., using 4.2 ml of a ficin solution with an enzymatic activity of 45 μUPABA/ml for cell detachment. Sub-culture was carried out according to the following scheme: D7: cell inoculation by ratio 1/8 in 100 ml of growth medium composed of 12.5% of conditioned medium; D12: cell inoculation by ratio 1/8 in 100 ml of growth medium composed of 12.5% of conditioned medium; D17: cell inoculation by ratio 1/4 in 100 ml of growth medium composed of 25% of conditioned medium; D21: repeat the scheme starting at D7. In this procedure, the cell inoculum is not fixed to a targeted cell density. Cell countings, carried out for control, showed that the cell inoculum densities were between 8000 cells/cm² and 33000 cells/cm². The MRC-5 cells reached the Population Doubling Level 71 after 90 days of culture with a cell growth rate of 0.56 PDL/day after which senescence was observed. These results, illustrated in FIGS. 1, 2 and 3, were equivalent to what is observed with a procedure using porcine trypsin for cell detachment and bovine serum (senescence at around PDL 65 after 83 days and cell growth rate of around 0.55 PDUday (Wistrom C, Villeponteau. B. Exp. Gerontol, 1990; 25(2): 97-105)).

### Example 6

### MRC-5 Cell Culture Until Senescence Using Bromelain Protease for Cell Detachment

This process was similar to the one disclosed in the Example 4 except for the following points: a bromelain solution with an enzymatic activity of 0.01105 Gelatin Digested Units (GDU)/ml was used for cell detachment instead of the ficin solution.

Sub-culture was carried out according to the following scheme: D7: cell inoculation by ratio 1/8 in 100 ml of growth medium composed of 12.5% of conditioned medium; D12: cell inoculation by ratio 1/8 in 100 ml of growth medium composed of 12.5% of conditioned medium; D17: cell inoculation by ratio 1/4 in 100 ml of growth medium composed of 12.5% of conditioned medium; and D21: repeat the scheme starting at D7.

Cell countings, carried out for control, showed that the cell inoculum densities were between 8000 cells/cm² and 33000 cells/cm². The MRC-5 cells reached the Population Doubling Level 67 after 82 days of culture with a cell growth rate of 0.56 PDL/day after which senescence was observed. These results, as illustrated in FIGS. 1, 2 and 3, were equivalent to what is observed with a procedure using porcine trypsin for cell detachment and bovine serum (senescence at PDL 65 after 83 days and cell growth rate=0.55 PDL/day (Wistrom C, Villeponteau. B. Exp. Gerontol, 1990; 25(2): 97-105)).

### Example 7

### HAV Production on MRC-5 Cells Multiplied by Using Ficin Protease for Cell Detachment

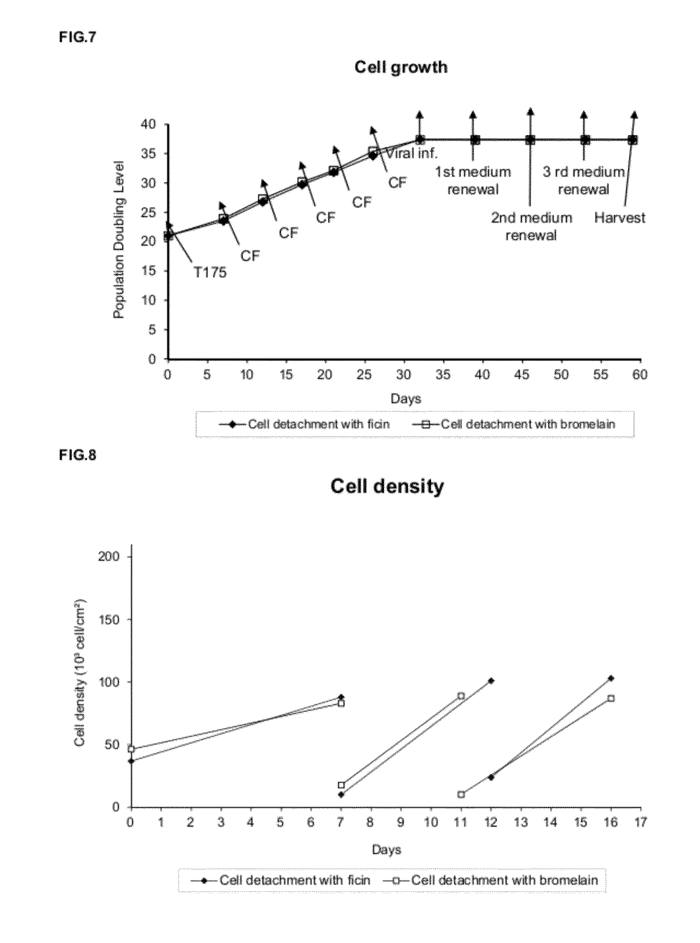

HAV production in Nunc Cell Factories (CF) with MRC-5 cells cultured by using ficin protease for cell detachment, requires the implementation of the method describe in the steps 5 to 7 of Example 3. MRC-5 cells coming from a cell bank (at PDL 21) free of animal-origin components are multiplied in Nunc T175 cm² flask then in CF until the Population Doubling Level 36 is reached, by using the method describe in the steps 1 to 3 of the Example I (FIG. 7). MRC-5 cells are infected with HAV stock seed prepared in the medium described in the Table 2 at a target MOI of 0.01. After infection, cells are incubated at 32° C. during 27 days with 3 medium renewals after 7, 14 and 21 days (FIG. 7). HAV harvest is carried out 27 days after infection by starting with two washings of the cell layer with PBS, then by detaching cells with a PBS supplemented with about 0.2 g/L of EDTA and finally by freezing cells. Antigenic titers of the HAV bulk obtained using this procedure are between 250 and 350 E.L.I.S.A Units (ELU)/0.1 ml. These results are equivalent to what is observed with a procedure using porcine trypsin for cell detachment and bovine serum (HAV Bulk antigenic titers=250 ELU/0.1 ml).

### Example 8

### HAV Production on MRC-5 Cells Multiplied by Using Bromelain Protease for Cell Detachment

This process is similar to the one disclosed in the Example V except that a bromelain solution with an enzymatic activity of 0.01105 Gelatin Digested Units (GDU)/ml is used for cell detachment instead of the ficin solution.

Antigenic titers of the HAV bulk obtained using this procedure are between 250 and 350 E.L.I.S.A Units/0.1 ml. These results are equivalent to what is observed with a procedure using porcine trypsin for cell detachment and bovine serum (HAV Bulk titer 250 ELU/0.1 ml).

### Example 9

### Cell Banking of MRC-5 Cell Multiplied by Using Ficin Protease for Cell Detachment

A cell banking procedure for MRC-5 cells using ficin repeated the cell production methods with the being process free of components from animal origin as described in the steps 1 to 3 of Example 3, until the chosen PDL is reached (PDL 21). At this PDL, cells were frozen following the method described in the step 4 of Example 3. MRC-5 cells coming from a cell bank (at PDL 14) containing serum were thawed, inoculated in a Nunc T175 cm² flask with 100 ml of the medium described in Table 2 and incubated at 37° C. After seven days, sub-cultures (see steps 1 to 3) were carried out in Nunc T-175 cm² flask at 37° C., using 4.2 ml of a ficin solution with an enzymatic activity of 45 pUPABA/ml for cell detachment. Sub-culture was carried out according to the following scheme: D7: cell inoculation by ratio 1/8 in 100 ml of growth medium composed of 12.5% of conditioned medium; D12: cell inoculation by ratio 1/4 in 100 ml of growth medium composed of 25% of conditioned medium; D16: cell banking using a ratio 1/4.

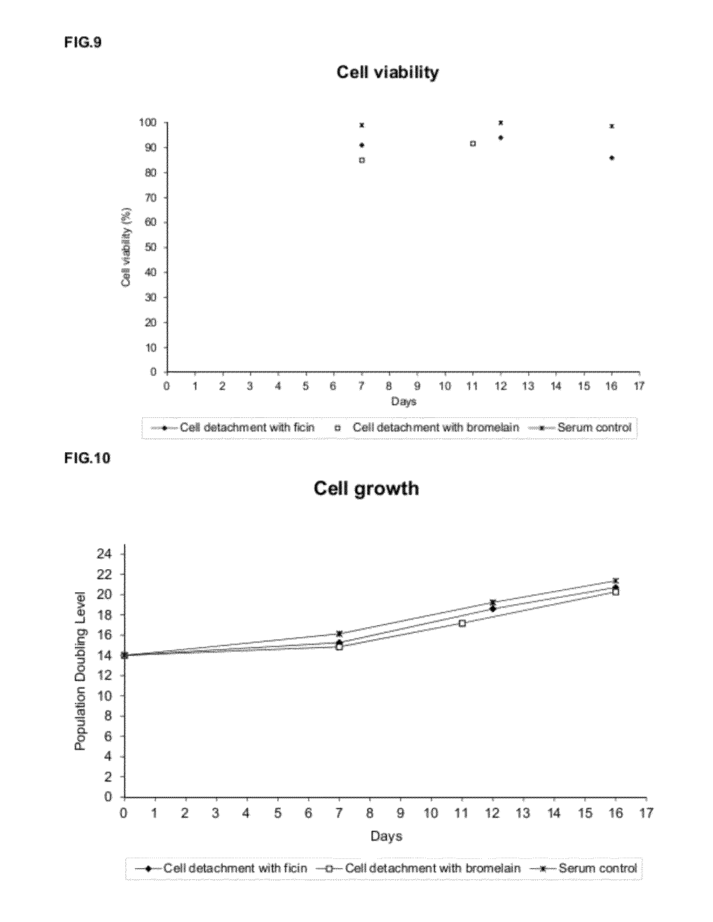

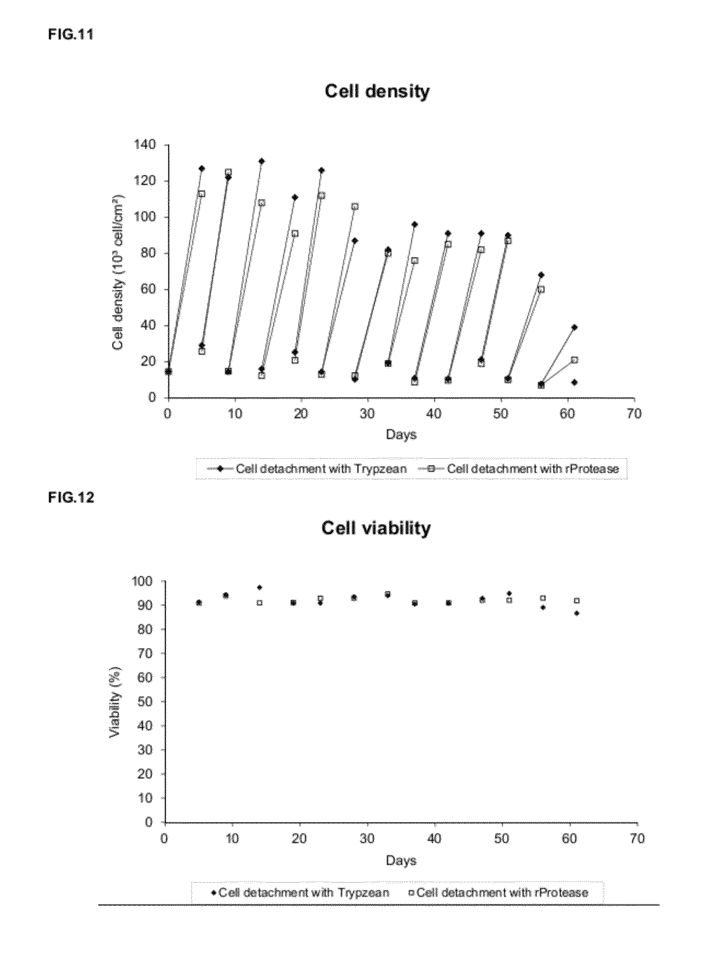

In this procedure, the cell inoculum was not fixed to a targeted cell density. The results are shown in FIGS. 8, 9 and 10. The MRC-5 cells reached the PDL 21 after 16 days. At this PDL MRC-5 cells were frozen in the medium free of animal-origin components supplemented with 7.5% DMSO and 0.1% of methylcellulose. After thawing, these MRC-5 cells showed a viability and a cell growth equivalent to what is observed before freezing (viability of about 90-95% and cell growth rate >0.55 PDL/day) (see FIGS. 1, 2 and 3). These results are equivalent to what is observed with a procedure using porcine trypsin for cell detachment and bovine serum (viability of about 90-95% and cell growth rate=0.55 PDUday (Wistrom C, Villeponteau. B. Exp. Gerontol, 1990; 25(2): 97-105)).

### Example 10

### Cell Banking of MRC-5 Cell Multiplied by Using Bromelain Protease for Cell Detachment

This process is similar to the one disclosed in Example 9 except for the following: a bromelain solution with an enzymatic activity of 0.01105 Gelatin Digested Units (GDU)/ml is used for cell detachment instead of the ficin solution. Sub-culture are carried out according to the following scheme: D7: cell inoculation by ratio 1/4 in 100 ml of growth medium composed of 25% of conditioned medium; D11: cell inoculation by ratio 1/8 in 100 ml of growth medium composed of 12.5% of conditioned medium; D16: cell banking using a ratio 1/4.

Results are shown in FIGS. 8, 9 and 10. The MRC-5 cells reached the Population Doubling Level 21 after 16 days. After thawing, these MRC-5 cells showed a viability and a cell growth equivalent to what is observed before freezing (viability of about 90-95% and cell growth rate >0.55 PDUday) (see FIGS. 1, 2 and 3). These results are equivalent to what is observed with a procedure using porcine trypsin for cell detachment and bovine serum (viability of about 90-95% and cell growth rate 0.55 PDL/day (Wistrom C, Villeponteau. B. Exp. Gerontol, 1990; 25(2): 97-105)).

### Example 11

### MRC-5 Cell Culture Until Senescence Using Trypzean or rProtease for Cell Detachment

A small scale procedure for MRC-5 cell senescence testing was carried out, repeating the cell production method with the process free of animal-origin components described in the steps 1 to 3 of Example 3, until senescence was observed. MRC-5 cells from a cell culture around PDL 27, free of components from animal origin, were propagated in Nunc T175 cm² using a Trypzean solution with an activity of 1 USP/ml or using a rProtease (Invitrogen) solution (stock solution 30 time diluted in PBS supplemented with EDTA as used for cell detachment, see step 1 Example 3), according to the following scheme: D0: cell inoculation by ratio 1/8 in 100 ml of growth medium composed of 12.5% of conditioned medium; D5: cell inoculation by ratio 1/4 in 100 ml of growth medium composed of 25% of conditioned medium; D9: cell inoculation by ratio 1/8 in 100 ml of growth medium composed of 12.5% of conditioned medium; D14: repeat the scheme starting at D0.

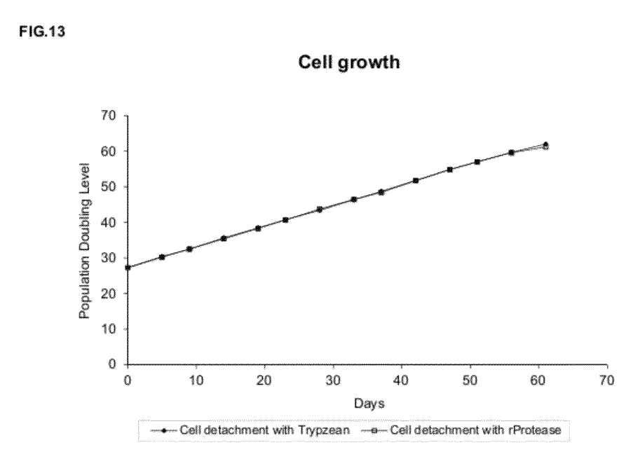

In this procedure, the cell inoculum was not fixed to a targeted cell density. Cell countings, carried out for the control sample, showed that the cell inoculum densities were between 8000 cells/cm² and 30000 cells/cm². MRC-5 cells reached a PDL superior to 60 after 61 days of culture with a cell growth rate of around 0.56 PDL/day after which senescence was observed. These results, illustrated in FIGS. 11, 12 and 13, are equivalent to what is observed with a procedure using porcine trypsin for cell detachment and bovine serum (senescence at around PDL 65 after 83 days and cell growth rate of around 0.55 PDL/day (Wistrom C, Villeponteau. B. Exp. Gerontol, 1990; 25(2): 97-105)).

### Example 12

### Use of Ficin Protease for Cell Detachment in Comparison to Trypsin (Porcine) and Trypsin-Like-Enzyme (Tryple; Recombinant Trypsin)

The methods of the invention for passaging mammalian cell lines were tested in Vero cells, which are derived from kidney epithelial cells of the African Green Monkey. These cells were cultured in a medium of the invention as described in the above examples.

Cell detachment for cell passaging was performed in a comparative setting using three different proteases: ficin, trypsin-like-enzyme (TrpLE-recombinant) and procine trypsin, which acted as a comparative control to assess the activity of the non-animal proteases.

The following steps were performed when using ficin to passage the cell culture. The medium was discarded from the flask containing the cell culture. The cell layer was rinsed with 15 ml of washing buffer [PBS without Ca, Mg pH 7.4, EDTA 0.54 mM]. 5 ml of ficin in solution [Ficin with PBS, glucose 0.5 g/L, EDTA 0.1 g/L, cysteine 1.0908 g/L] was added. The mixture was incubated for a maximum of 20 minutes at room temperature, and the cell suspension was harvested and directly diluted in the cell culture medium to inactivate the ficin. Cells were grown for another cell cycle up to confluence.

The following steps were performed when using the porcine trypsin to passage the Vero cells. The medium was discarded from the flask containing the cell culture. 50-90 ml of trypsin in solution was added to the flask, and the mixture was incubated for a maximum of 10 minutes at room temperature. The cell suspension was harvested and directly diluted in cell culture medium to inactivate the enzyme. Cells were grown for another cell cycle up to confluence.

The following steps were performed when using recombinant TrpLE to passage the Vero cells. Prior to cell passaging, a soybean trypsin inhibitor (STI) solution (10000 USP/ml) was diluted in the animal free cell culture medium (125 ml STI in 875 ml medium). The medium was discarded and the cell layer was rinsed with 30 ml of PBS [without Ca, Mg pH 7.4 EDTA 0.54 mM]. 6 ml (0.04 rPU/ml) of TrpLE was added and the mixture incubated at 37° C. for approximately 20-30 min. The cell suspension was harvested and diluted in the STI solution. Cells were grown for another cell cycle up to confluence.

The above-described passaging methods were carried out on the Velo cells for three consecutive cell passages, beginning with cell passage 133. The impact of the use of the different enzymes was observed for indicators of cell viability including cell necrosis, late and early apoptosis, and overall viability. These results are shown in table 4.

TABLE 4

| | Necrosis | Late apoptosis | Viability | Early apoptosis |
|---|---|---|---|---|
| | (%) | (%) | (%) | (%) |
| P133 | | | | |
| Ficin | 10.4 | 2.0 | 81.7 | 5.9 |
| trypsin | 11.5 | 3.2 | 78.7 | 6.7 |
| TrpLE | 25.0 | 2.2 | 68.7 | 4.1 |
| P134 | | | | |
| Ficin | 4.1 | 4.3 | 82.8 | 8.8 |
| trypsin | 3.2 | 5.4 | 74.3 | 17.1 |
| TrpLE | 4.9 | 24.0 | 60.8 | 10.3 |
| P135 | | | | |
| Ficin | 9.0 | 6.4 | 72.9 | 11.7 |
| trypsin | 10.3 | 9.2 | 59.7 | 20.7 |
| TrpLE | 9.2 | 21.7 | 57.9 | 11.2 |

The results demonstrate an improved performance using the ficin as compared to both recombinant TrpLE and the porcine trypsin.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims. In the claims that follow, unless the term “means” is used, none of the features or elements recited therein should be construed as means-plus-function limitations pursuant to 35 U.S.C. §112, ¶6.

What is claimed is:

1. A method of establishing an animal anchorage-dependent cell culture, comprising:
seeding animal cells in a culture medium which is devoid of exogenous components of primary and secondary animal origin, and which comprises exogenous components of non-animal origin comprising:
iv) at least one growth factor of non-animal origin selected from EGF, FGF, tri-iodo-L-tyronine and hydrocortisone;
v) at least one exogenous growth factor of non-animal origin selected from the group consisting of IGF-1 and/or insulin; and
passaging said cell culture with a protease of non-animal origin.

2. The method of claim 1, wherein the protease is a cysteine endopeptidase.

3. The method of claim 2, wherein the cysteine endopeptidase is ficin, stem bromelain, or actinidin.

4. The method of claim 2, wherein the cysteine endopeptidase is ficin.

5. The method of claim 1, wherein the protease is a neutral fungal protease, a neutral bacterial protease or a trypsin-like protease.

6. The method of claim 1, wherein the culture medium further comprises a hydrolysate on non-animal origin.

7. The method of claim 6, wherein the hydrolysate is wheat hydrolysate.

8. The method of claim 1, wherein the anchorage-dependant cells are AGMK, VERO, MDCK, CEF or CHO cells.

9. The method of claim 1, wherein said cell culture is used for the production of viruses.

10. The method of claim 1, wherein the cell culture medium comprises conditioned medium.

11. The method of claim 1, wherein the cell culture medium comprises fresh medium.

12. A method for establishing a culture comprising animal diploid cells, comprising:
d) seeding the cells in a culture device comprising an adhesion support and a culture medium comprising:
j) at least one growth factor of non-animal origin selected from EGF, FGF, tri-iodo-L-tyronine and hydrocortisone;
iv) at least one exogenous growth factor of non-animal origin selected from the group consisting of IGF-1 and/or insulin, and
v) a wheat protein hydrolysate;
e) allowing the cells to adhere to the substrate;
f) maintaining the cells for a desired number of cell divisions;
d) dissociating cells from the substrate with a protease of non-animal origin, thereby forming a cell suspension; and
e) placing the cell suspension and a cell culture medium of step a) in a culture device comprising an adhesion support.

13. The method of claim 12, further comprising harvesting the conditioned medium resulting from step a), and using the conditioned media alone or in combination in step e).

14. The method of claim 12, wherein steps b) to e) are repeated two or more times.

15. The method of claim 12, wherein the cells dissociated in step d) are harvested and frozen to produce a cell bank.

16. The method of claim 15, wherein steps b) to d) are repeated two or more times before the cells are harvested to be frozen.

17. The method of claim 12, wherein the protease used in step d) is inactivated after treatment.

18. A method for maintaining an animal cell culture, said method comprising:
a) providing a culture medium as herein defined to animal cells adhered to a substrate;
b) maintaining the cells for a desired number of cell divisions;
c) dissociating cells from the substrate with a protease of non-animal origin, thereby forming a cell suspension; and
d) placing the cell suspension and a cell culture medium of step a) on a new substrate.

19. The method of claim 18, wherein steps a) to d) are repeated two or more times.

20. The method of claim 18, wherein the cells dissociated in step c) are harvested and frozen to produce a cell bank.

21. The method of claim 20, wherein steps b) to d) are repeated two or more times before the cells are harvested to be frozen.

22. The method of claim 18, wherein the protease used in step c) is inactivated after treatment.

23. A composition comprising
a culture medium comprising
a) at least one growth factor of non-animal origin selected from EGF, FGF, tri-iodo-L-tyronine and hydrocortisone;
b) at least one exogenous growth factor of non-animal origin selected from the group consisting of IGF-1 and/or insulin, and
c) a wheat protein hydrolysate; and
diploid anchorage-dependent animal cells.
